(19) United States
(12) Patent Application Publication
(10) Pub. No.: US 2003/0228635 A1
(43) Pub. Date: Dec. 11, 2003

(54) Cell proliferation assays and methods

(75) Inventors: Huaizhong Hu, Madison, WI (US); Alice Puchalski, Madison, WI (US)

Correspondence Address: MEDLEN & CARROLL, LLP, Suite 350, 101 Howard Street, San Francisco, CA, 94105, US

(73) Assignee: Renovar, Inc., Madison, WI

(21) Appl. No.: 10/367,494

(22) Filed: Feb. 14, 2003

Related U.S. Application Data
(60) Provisional application No. 60/358,003, filed on Feb. 15, 2002.

(51) Int. Cl. A61K 39/395; G01N 33/53; G01N 33/567

(52) U.S. Cl. 435/7.2; 424/178.1

(57) ABSTRACT

The present invention relates to compositions and methods for conducting cell division and proliferation assays (e.g., using flow cytometry), and in particular, to improvements in the effective storage and handling of dyes and reagents used therein.

The present invention claims priority to U.S. Provisional Application No. 60/358,003, filed Feb. 15, 2002, the entire contents of which are incorporated herein by reference.

### FIELD OF THE INVENTION

The present invention relates to compositions and methods for conducting cell division and proliferation assays (e.g., using flow cytometry), and in particular, to improvements in the effective storage and handling of dyes and reagents used therein.

### BACKGROUND OF THE INVENTION

The rapid and accurate analysis of cell viability and proliferation is an important feature of many cell biology and drug-discovery research efforts. For example, cell proliferation analysis can provide important information as to the effects of growth factors, chemotactic, metastatic, and apoptotic agents, and on certain aspects of immunological research. Accordingly, analysis of cell proliferation and the characterization of agents that either promote or retard cell proliferation are intensely studied areas in biotechnology.

Cell proliferation is a measurement of the number of cells that are dividing in a culture. One way of measuring this parameter is by performing clonogenic assays. In these assays, a defined number of cells are plated onto an appropriate matrix for cell growth. After a set period of time the number of colonies that formed are counted.

One drawbacks to clonogenic assay techniques is that they are tedious to perform and impractical for large numbers of samples. In addition, if cells divide only a few times and then become quiescent, colonies may be too small to be counted and the number of dividing cells will thus be underestimated.

Another technique for analyzing cell proliferation is by measuring DNA synthesis. Typically, in these types of assays labeled DNA precursors (e.g., ³Hthymidine or bromodeoxyunridine) are added to the cells and their subsequent incorporation into DNA is quantitated after incubation. The amount of labeled precursor incorporated into DNA is quantified by measuring the total amount of labeled DNA in a population, or by detecting the labeled nuclei microscopically. Incorporation of the labeled precursor into DNA is directly proportional to the amount of cell divisions occurring in the culture.

Analysis of cell proliferation can also be measured using more direct techniques. Generally these techniques require the addition of molecules that regulate the cell cycle which are subsequently measured either by their activity (e.g., CDK kinase assays) or by quantifying their amounts (e.g., Western Blot, ELISA, or immunohistochemistry).

Another direct technique for analyzing cell proliferation involves staining the cells of interest in vivo or in vitro with fluorescent dyes. Flow cytometry is subsequently used to discriminate proliferating cells. For example, flow cytometry techniques have been developed for the analysis of lymphocyte proliferation by the serial halving of the fluorescence intensity of vital dyes (e.g., 5(6)-carboxyfluorescein diacetate, succinimidyl ester “CFSE”). This technique allows for the in vivo and in vitro visualization and analysis of from 8 to 10 discrete cycles of cell division.

What is needed are more convenient and cost effective, compositions and methods for conducting cell proliferation assays, and in particular, cell proliferation assays using fluorescent dyes.

### SUMMARY OF THE INVENTION

The present invention relates to compositions and methods for conducting cell division and proliferation assays (e.g., using flow cytometry), and in particular, to improvements in the effective storage and handling of dyes and reagents used therein.

In certain embodiments, the present invention provides a kit for conducting cell proliferation assays comprising a container having an aliquot of a fluorescein dye (e.g., CFSE dye) at a concentration of from about 0.01 mM to about 10 mM, from about 0.1 mM to about 5 mM, and from about 0.8 to about 2 mM. In preferred embodiments, the fluorescein dye concentration is about 0.8 mM to about 1 mM.

In other embodiments, the fluorescein dye concentration is at least about 0.01 mM. In still other embodiments, the fluorescein dye concentration is at least about 0.5 mM. In preferred embodiments, the fluorescein dye concentration is at least about 1 mM.

The present invention is not intended to be limited, however, to providing aliquots CFSE dye at a concentration of from about 0.01 mM to about 10 mM. Likewise, the present invention is not intended to be limited to providing kits comprising CFSE dyes, as a number of suitable other fluorescein dyes are known in the art and are recited herein.

In some embodiments, the kits of the present invention further comprise one or more conjugated antibodies (e.g., R-phycoerythrin conjugated antibodies). In still other embodiments, the kits further comprise instructions for using said fluorescein dye (e.g., CFSE dye) in a cell proliferation assay experiment.

In still other embodiments, the kits of the present invention further comprise two or more different conjugated antibodies (e.g., R-phycoerythrin conjugated). In certain of these embodiments, the R-phycoerythrin conjugated antibodies are selected from the group consisting of R-PE anti IgG, R-PE anti CD3, R-PE anti CD4, R-PE anti CD8, and R-PE anti CD19.

In preferred embodiments, the compositions and methods disclosed herein are suitable for performing cell proliferation assays. In certain embodiments kits are provided one or more containers containing one or more aliquots of reagents, cell culture media, and buffers, etc. suitable for a for a single cell proliferation assay. In certain other embodiments, these containers contain an aliquot of reagent, cell culture media, and buffers, etc. suitable for two or more cell proliferation assays. In still other embodiments, the present invention contemplates providing a plurality of containers for any or more of the particular reagents, cell culture mediums, and buffers, etc., provided in the cell proliferation assay experiment kits of the present invention.

In some embodiments of the present invention, the kits further comprise one or more components for detecting intracellular compounds of interest (e.g., cytokines [e.g., interleukins, such as: IL-2; -3; -4; -5; -6; -7; -8; -9; -10; -11; -12; -13; -15; and -16; etc.], chemokines [e.g., MCAF, MIP-1α, MIP-1β; RANTES; IL-8, PK-4, NAP-2, etc.], TNF-α, and IFN-γ, etc.). In some embodiments, the intercellular detecting is conducted by providing, and subsequently detecting, one or more labeled (e.g., fluorescence, radioactivity, etc.) antibodies that bind to the intercellular target compound of interest. In other embodiments, the intercellular detecting is conducted by providing, and subsequently detecting, one or fluorescent dye compounds that bind to the intercellular target compound of interest.

In some embodiments, the kits of the present invention further comprise a container having an aliquot of propidium iodide staining solution. It is contemplated that in certain of these embodiments, these kits contain an aliquot of propidium iodide staining solution suitable for about 20 or more cell proliferation assay experiments.

In still further embodiments, the kits of the present invention further comprise a container having an aliquot of phytohemagglutinin.

In yet other embodiments, the kits of the present invention further comprise a container having an aliquot of concanavalin A.

Particularly preferred embodiments of the present invention provide compositions that are stable for long periods of time when stored as directed under the proper conditions (e.g., temperature, humidity, light, etc.). In some embodiments, the kits disclosed herein provide reagents for cell proliferation assay experiments that are stable for at least 1 to 12 or more months. In preferred embodiments, the pre-aliquoted CFSE portions are stable for at least 3 to 9 months. In particularly preferred embodiments, the pre-aliquoted CFSE portions are stable for at least 4 to 8 months.

In yet another embodiment, the present invention provides a kit for conducting cell proliferation assays comprising: a) a first container having an aliquot of first fluorescein dye, wherein the fluorescein dye (e.g., CFSE) at a concentration of from 0.01 mM to about 10 mM, and b) a second container having an aliquot of second fluorescein dye. In certain of these embodiments, the kits further comprise instructions for using the first fluorescein dye (e.g., CFSE dye) and the second fluorescein dye in a cell proliferation assay experiment.

In certain embodiments, the present invention provides a kit for conducting cell proliferation assays comprising a container having an aliquot of a fluorescein dye (e.g., CFSE dye) at a concentration of from about 0.01 mM to about 10 mM, from about 0.1 mM to about 5 mM, and from about 0.8 to about 2 mM. In preferred embodiments, the fluorescein dye concentration is about 0.8 mM to about 1 mM.

In other embodiments, the fluorescein dye concentration is at least about 0.01 mM. In still other embodiments, the fluorescein dye concentration is at least about 0.5 mM. In preferred embodiments, the fluorescein dye concentration is at least about 1 mM. Certain of these embodiments, provide an aliquot of CFSE dye that contains an amount of the CFSE dye suitable for a single cell proliferation assay experiment.

Additional related embodiments, further comprise one or more R-phycoerythrin conjugated antibodies selected from the group consisting of R-PE anti IgG, R-PE anti CD3, R-PE anti CD4, R-PE anti CD8, and R-PE anti CD19. Still other related embodiments of the present invention further comprises a container having an aliquot of propidium iodide staining solution. Yet another embodiment of the present invention further comprises a container having an aliquot of phytohemagglutinin. The present invention contemplates providing a container having an aliquot of concanavalin A in still other related embodiments.

The present invention also provides a kit for conducting cell proliferation assays comprising: a) a first container having an aliquot of first fluorescein dye, wherein the fluorescein dye comprises CFSE dye at a concentration of from about 0.01 to about 10 mM, b) a second container having an aliquot of a second fluorescein dye, and c) one or more different conjugated antibodies (e.g., R-phycoerythrin conjugated antibodies). In certain of these embodiments, the kits further comprise instructions for using the first CFSE dye and the second fluorescein dye in a cell proliferation assay experiment. In some of these embodiments, the present invention provides an aliquot of CFSE dye with an amount of the CFSE dye suitable for a single cell proliferation assay experiment.

In certain embodiments, the present invention provides a kit for conducting cell proliferation assays comprising a container having an aliquot of a fluorescein dye (e.g., CFSE dye) at a concentration of from about 0.01 mM to about 10 mM, from about 0.1 mM to about 5 mM, and from about 0.8 to about 2 mM. In preferred embodiments, the fluorescein dye concentration is about 0.8 mM to about 1 mM.

In other embodiments, the fluorescein dye concentration is at least about 0.01 mM. In still other embodiments, the fluorescein dye concentration is at least about 0.5 mM. In preferred embodiments, the fluorescein dye concentration is at least about 1 mM.

In still other embodiments, the kits of the present invention provide two or more R-phycoerythrin conjugated antibodies selected from the group consisting of R-PE anti IgG, R-PE anti CD3, R-PE anti CD4, R-PE anti CD8, and R-PE anti CD19.

Certain embodiments of the present invention provide a composition comprising a container having an aliquot of fluorescein dye (e.g., CFSE dye) at a concentration of from about 0.01 mM to about 10 mM , wherein the aliquot of CFSE dye contains an amount of dye suitable for a single cell proliferation assay experiment, and further wherein the kit is stable for at least 7 months.

Still further embodiments of the present invention provide a method of data analysis comprising: a) providing data from a flow cytometry assay experiment, wherein the data is displayed such that data corresponding to test cells are distinguishable from data corresponding to background cells, b) selecting a subset of the data from flow cytometric assay experiment such that the subset contains substantially all of the data corresponding to test cells and excludes substantially all of the data corresponding to the background cells; and c) analyzing the subset to provide a cell proliferation result.

In certain of these embodiments, the analyzing further comprises preparing one or more user defined gated sets of the data from a flow cytometry assay experiment, wherein the one or more user defined gated sets of the data from a flow cytometry assay experiment comprises a first, second, third, and fourth, etc., regions. The present invention is not intended to be limited, however, to any particular number or orientation of user selected regions. In further related embodiments, the analyzing further comprises determining the ratio of signal in one region compared to one or more other regions.

### DESCRIPTION OF THE FIGURES

FIGS. 1A-1H show exemplary data displays of the present invention.

FIGS. 2A-2E show exemplary data displays of the present invention.

Definitions

To facilitate an understanding of the present invention, a number of terms and phrases are defined below.

As used herein, the term “host cell” refers to any cell (e.g., eukaryotic cells such as mammalian cells, avian cells, amphibian cells, plant cells, fish cells, and insect cells), whether located in vitro or in vivo. In the present invention, “cells of interest” are host cells that the user has designated as being the subject of the flow cytometry analysis techniques disclosed herein. Furthermore, as used herein, the cells of interest can be in vivo, in vitro, or ex vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

The term “microorganism” as used herein means an organism too small to be observed with the unaided eye and includes, but is not limited to bacteria, virus, protozoans, fungi, and ciliates.

The term “bacteria” refers to any bacterial species including eubacterial and archaebacterial species.

The term “multi-drug resistant” or multiple-drug resistant” refers to a microorganism which is resistant to more than one of the antibiotics or antimicrobial agents used in the treatment of said microorganism.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

The terms “label” or “marker” as used herein refer to any atom or molecule that can be used to provide a detectable (preferably quantifiable) signal. Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral.

The term “sample” in the present specification and claims is used in its broadest sense. On the one hand it is meant to include a specimen or culture (e.g., animal tissues or fluids, or microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, birds, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

As used herein, the term “purified” or “to purify” refers to the removal of contaminants from a sample.

### DESCRIPTION OF THE INVENTION

The present invention relates to compositions and methods for conducting cell division and proliferation assays (e.g., using flow cytometry), and in particular, to improvements in the effective storage and handling of dyes and reagents used therein.

The present invention provides a simple and sensitive technique for the analysis of multiple cell parameters. The present invention contemplates that the compositions and methods of the present invention provide several advantages over existing methods for determining cell proliferation. For example, one commonly used method for studying cell proliferation requires the use of ³H thymidine. However, the ³H thymidine method requires the handling of radioactive materials, and fails to provide positive identification of the population of proliferating cells. (See e.g., Karube T., et al., “A new immunocytochemical method to measure cell kinetics of human lymphocytes by using monoclonal antibody against bromodeoxyuridine,” Nippon Ketsueki Gakkai Zasshi—Acta Haematologica Japonica, 50:862 (1987); Gratzner, H. G., and R. C. Leif, “An immunofluorescence method for monitoring DNA synthesis by flow cytometry,” Cytometry, 1:385 (1981)). In contrast, preferred embodiments of the present invention do not require the use of radioactive isotopes.

Another commonly used method for studying cell proliferation is based on bromodeoxyuridine (BrdU) incorporation. This assay method is limited, however, in that it does not permit the user to distinguish the various rounds of cell division and proliferation. Since, detection of BrdU is accomplished using anti-BrdU antibodies, the user is required to permeabilize the cells of interest thus precluding the use of PI to identify live cells. In fact, neither of the cell proliferation assay techniques described above allow viable cells to be recovered. In contrast, the compositions and methods of the present invention allow viable cells to be recovered for further analysis. Indeed, the flow cytometry techniques employed in preferred embodiments of the present invention permit the study of specific populations of proliferating cells and the identification of about 7-10 successive cell generations. In particularly preferred embodiments, the flow cytometry techniques disclosed herein are combined with the concomitant use of labeled (e.g., fluorescence) antibodies and propidium iodide (PI) which further facilitates the assessment of cell viability and cellular phenotypes.

One of the contemplated applications of the present invention includes, but is not limited to, the analysis of antigen specific and non-specific T cell proliferation. (See e.g., Angulo R., D. A. Fulcher, “Measurement of Candida-specific blastogenesis: Comparison of carboxyfluorescein succinimidyl ester labelling of T cells, thymidine incorporation, and CD69 expression,” Cytometry, 34:143 (1998); Lyons, A. B., “Analysing cell division in vivo and in vitro using flow cytometric measurement of CFSE dye dilution,” J. Immunol. Meth., 243:147 (2000) each herein incorporated by references in its entirety). Briefly, in certain of these embodiments, lymphocytes are incubated with membrane permeable, non-fluorescent CFSE which passively diffuses into cells. Excess dye is then washed away and any quiescent cells are induced to proliferate by in vitro mitogenic or antigenic stimulation. The cells are maintained in culture for a sufficient period of time to allow for cell proliferation, typically, about four days. In preferred embodiments, the subsequent staining of newly proliferated cells with labeled antibodies (e.g., fluorescence) for various cell surface molecules allows the user to determine the proliferation of specific lymphocyte subsets.

1. Kits

One of the objects of the present invention is to provide cell proliferation assay kits comprising all or the majority of the necessary components for conducting cell proliferation assays in conveniently sized aliquots. The present invention contemplates that providing one or more premeasured aliquots of key cell proliferation assay reagents (e.g., fluorescein dye) will reduce reagent waste thus increasing cost effectiveness and value. In particular, the present invention contemplates providing aliquoted portions of the fluorescein compounds required in cell proliferation assays using flow cytometry techniques.

In preferred embodiments, the compositions and methods of the present invention are provided in a convenient kit form with all necessary reagents, media, and buffers, including, but not limited to, fluorescein dyes (e.g., carboxy-fluorescein diacetate, succinimidyl ester), cell activators (e.g., T cell activator such as phytohemagglutinin (PHA)), a nucleic acid stain (e.g., propidium iodide (PI)), one or more R-phycoerythrin (R-PE) conjugated antibodies (e.g., R-PE anti IgG, R-PE anti CD3, R-PE anti CD4, R-PE anti CD8, and R-PE anti CD19), cell culture media, necessary buffers, antibiotics, and optionally, one or more a mitosis arrestors. In certain of these embodiments, the nucleic acid stain provided in the kit is capable of staining the DNA of dead cells or cells in late stages of apoptosis. The present invention is not limited, however, to providing kits comprising all of the reagents, cell culture mediums, and buffers, etc., necessary for conducting flow cytometry cell proliferation assays. Indeed, in some preferred embodiments, kits are provided without one or more of the reagents, culture media, mitosis arrestors, buffers, etc. necessary for conducting flow cytometry cell proliferation assays. In certain of these embodiments, it is contemplated that the kit user will be able to easily tailor the reactions conditions and reagents to their particular needs.

In preferred embodiments of the present invention, kits are provided with pre-aliquoted amounts of one or more reagents, buffers and media. In particularly preferred embodiments, kits are provided with one or more pre-aliquoted portions of one or more fluorescein dyes. The present invention is not limited, however, to any particular fluorescein dyes. Indeed, the present invention contemplates that any stable fluorescein dye may be supplied in pre-aliquoted portions for use with the flow cytometry kits disclosed herein.

In certain embodiments, one or more of the following compositions are additionally provided in the kits of the present invention. Any functional equivalents of the following components may alternatively be substituted for the components listed below. In deed, the present invention is not intended to be limited, however, to kits further comprising the following compositions. In certain embodiments, the kits of the present invention further comprise cell culture media (mediums). For example, certain kits may comprise pre-aliquoted quantities of RPMI 1640 with L-glutamine and 25 mM HEPES supplemented with 15% (v/v) fetal calf serum, antibiotic/antimycotic solution, non-essential amino acids, sodium pyruvate, and vitamin solution. In some embodiments, pre-aliquoted portions of Mitomycin C are provided when performing mixed lymphocyte reactions. It is contemplated that Mitomycin C is used in mixed lymphocyte reactions to arrest mitosis of stimulator cells so that only the proliferation of responder cells is measured. In certain of these embodiments, the suggested final concentration for inactivation of DNA synthesis is 400 μg/ml. Phosphate buffered saline (PBS) pH 7.2-7.4 is further provided in some embodiments as a multipurpose solution for washing and staining cells. As noted above, the present invention is not intended to be limited, however, to kits that provide the aforementioned, or any other, reagents, dyes, cell culture media, antibiotics, nutrients, mitosis arrestors, antibodies, buffers, etc.

In some embodiments of the present invention, the kits further comprise one or more components for detecting intracellular compounds of interest (e.g., cytokines [e.g., interleukins, such as: IL-2; -3; -4; -5; -6; -7; -8; -9; -10; -11; -12; -13; -15; and -16; etc.], chemokines [e.g., MCAF, MIP-1α, MIP-1 β; RANTES; IL-8, PK-4, NAP-2, etc.], TNF-α, and IFN-γ, etc.). In some embodiments, the intercellular detecting is conducted by providing, and subsequently detecting, one or more labeled (e.g., fluorescence, radioactivity, etc.) antibodies that bind to the intercellular target compound of interest. In other embodiments, the intercellular detecting is conducted by providing, and subsequently detecting, one or fluorescent dye compounds that bind to the intercellular target compound of interest.

In some embodiments, the kits disclosed herein further comprise instructions for conducting cell proliferation assays and/or for interpreting the data generated. In certain of these embodiments, the instructions supplied with the kits provides the user with information on the storage, handling, applications and use, of the kits or the individual reagents, culture media, and buffers contained therein. Instructions may be provided to the user in any form. For example, the present invention contemplates one or more of the following media forms for providing instructions: printed instructions (e.g., textual, photographic, and pictographic, etc.); digitally encoded instructions (e.g., instructions provide on computer-readable media); and instructions available via a communications network (e.g., World Wide Web, WANs, LANs, etc.) being provided on one or more computers processors or computer memories.

In some embodiments, for human in vivo use, instructions include regulatory agency (e.g., F.D.A.) mandated instructions, notices, and warnings.

In preferred embodiments, the types and quantities of the reagents, culture media, and buffers supplied in the disclosed kits are optimized to increase one or more of the following cell proliferation assay characteristics: stability, efficacy, purity, availability, ease of use, and/or economy and value.

2. Fluorescent Dyes

In preferred embodiments, one or more cells of interest are contacted with one or more detectable markers. In certain of these embodiments the detectable markers facilitate the discrimination of the labeled cells using flow cytometry techniques. In preferred embodiments, the detectable markers are fluorescent compounds. In certain of these embodiments the fluorescent markers comprise fluorescein molecules. In particularly preferred embodiments, the fluorescein molecules comprise one or more isomers of carboxy-fluorescein diacetate, succinimidyl ester (CFSE) having the following chemical formula: C₂₉H₁₉NO ₁₁. CFSE dye can be obtained from the any one of a number of commercial chemical supply houses (e.g., Fluka/Sigma-Aldrich Corp., St. Louis, Mo.).

CFSE consists of a fluorescein molecule containing a succinimidyl ester functional group and two acetate moieties. CFSE diffuses freely into cells where it is contemplated that intracellular esterases cleave the acetate groups thus converting (“activating”) the molecule into a fluorescent, membrane impermeable dye. Being membrane impermeable, the activated CFSE dye is not transferred to adjacent cells and is retained by the cell in its cytoplasm. CFSE does not adversely affect cellular function. (See e.g., Lyons, A. B., C. R. Parish, “Determination of lymphocyte division by flow cytometry,” J. Immunol. Meth. 171:131 (1994)). During each round of cell division, the relative intensity of the CFSE dye is decreased by half. This halving of dye intensity allows for the ready analysis of cell proliferation and the tracking of multiple cell generations based on the observed fluorescence.

The present invention is not limited, however, to any particular fluorescein dye or isomer of a particular fluorescein dye. In fact, a number of isomers of CFSE are contemplated for use in the present invention, as generally are other fluorescein dyes.

In preferred embodiments, the compositions and methods of the present invention are packaged in kits comprising pre-aliquoted amounts of CFSE. Prior to the present invention, cell proliferation study kits containing CFSE dye did not provide the user with pre-aliquoted portions of CFSE that were both stable and cost effective. The compositions and methods of the present invention provide kits comprising pre-aliquoted CFSE portions that are optimized for improved stability and cost effectiveness.

In some embodiments, the pre-aliquoted CFSE portions are stable for at least 1 to 12 or more months. In preferred embodiments, the pre-aliquoted CFSE portions are stable for at least 3 to 9 months. In particularly preferred embodiments, the pre-aliquoted CFSE portions are stable for at least 4 to 8 months. Appropriate storage conditions (e.g., temperature, humidity, exposure to light, etc.) for the individual reagents, including CFSE, comprising the kits disclosed herein per the manufacture's instructions for non-aliquoted materials.

In preferred embodiments, comprising pre-aliquoted amounts of CFSE, the amount and concentration of the aliquots are selected based upon considering and balancing 1) the stability of the aliquoted CFSE, 2) the minimum amount of CFSE in an aliquot necessary for effectively staining the cells of interest, and 3) the maximum amount of CFSE in an aliquot that the cells of interest can tolerate which also does not result in data acquisition problems (e.g., bleed over data). Prior to the present invention, the art did not teach the successful balancing of these competing considerations. The balancing of these consideration, in the present invention was worked out through empirical study and only after trial and error testing of a number of aliquot concentrations and amounts.

In certain embodiments, the present invention provides a kit for conducting cell proliferation assays comprising a container having an aliquot of a fluorescein dye (e.g., CFSE dye) at a concentration of from about 0.01 mM to about 10 mM, from about 0.1 mM to about 5 mM, and from about 0.8 to about 2 mM. In preferred embodiments, the fluorescein dye concentration is about 0.8 mM to about 1 mM.

In other embodiments, the fluorescein dye concentration is at least about 0.01 mM. In still other embodiments, the fluorescein dye concentration is at least about 0.5 mM. In preferred embodiments, the fluorescein dye concentration is at least about 1 mM.

The present invention contemplates that the conditions and pre-aliquoted amounts of reagents (e.g., CFSE) disclosed herein are useful for cell proliferation studies involving a wide range of cell types. In preferred embodiments, the present invention provides kits for conducting cell proliferation studies on immunological cell types (e.g., B- and T-cells) in both humans and rodents (e.g., mice, rats, Guinea pigs, etc.).

3. Data Acquisition

The flow cytometry techniques employed in preferred embodiments of the present invention permit the study of specific populations of proliferating cells and the identification of about 7-10 successive cell generations. In particularly preferred embodiments, the flow cytometry techniques and protocols disclosed herein are combined with the concomitant use of labeled (e.g., fluorescence) antibodies and propidium iodide (PI) which facilitates the assessment of cell viability and phenotype.

In preferred embodiments, flow cytometry data generated using the compositions and methods of the present invention is analyzed using analysis software capable of batch processing. In one preferred embodiment, the present invention contemplates using FLOWJO v3.6 (Tree Star, Inc., San Carlos, Calif.) flow cytometry data analysis software. The present invention further contemplates the novel adoption of certain batch processing features (e.g., gating) available in flow cytometry software programs for improved analysis of flow cytometry data. In certain of these embodiments, the present invention contemplates that manual partitioning (e.g., gating) of displayed (e.g., horizontally or vertically) data provides improved analysis of flow cytometry data, and in particular, improved accuracy in determining drug response/resistance in cells of interest. In a preferred embodiment the user manually partitions the data field into a first and second partition (e.g., regions) such that the partitioning step excludes substantially all of the background data and leaves substantially all of the data from cells of interest. In particularly preferred embodiments, the field of displayed flow cytometry data is manually partitioned by the user into a first, second, third, fourth, or more partitions (e.g., regions). In certain of these embodiments, the user partitions the data filed into an upper left, a lower left, an upper right, and a lower right quadrant. The present invention is not intended to be limited, however, to any particular number or orientation of user partitioned quadrants. The partitioned data field is then analyzed by determining the ratio of one or more regions in view of one or more other regions. However, it is understood that any mathematical formulas or operations necessary for arriving at a ratio of cells of interest to cells not of interest is within the scope of the present invention. In certain preferred embodiments, the upper left quadrant is divided by the sum of the upper left and upper right quadrants. Accordingly, in certain embodiments of the present invention, application of the above-mentioned partitioning formula for pre-screening flow cytometry data cuts down on unwanted signal, thus simplifying and improving flow cytometry data acquisition and analysis.

Certain embodiments of the present invention contemplate using compensation techniques in flow cytometry data acquisition and analysis to reduce the effects of fluorescent emission bleeding over when using multiple fluorescent dyes, and to reduce the effects of subtle emissions variations in dye isomers.

The following exemplary sample data obtained upon using the methods and compositions of the present invention were collected on a Becton Dickinson FACSCAN flow cytometer (Becton Dickinson, San Jose, Calif.). Data were analyzed and histograms generated using FLOWJO software (Tree Star, Inc., San Carlos, Calif.).

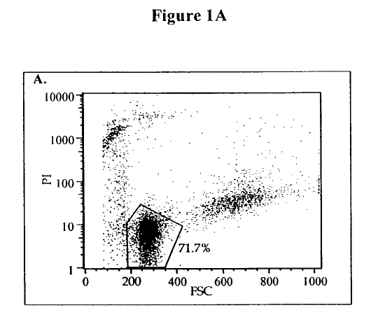

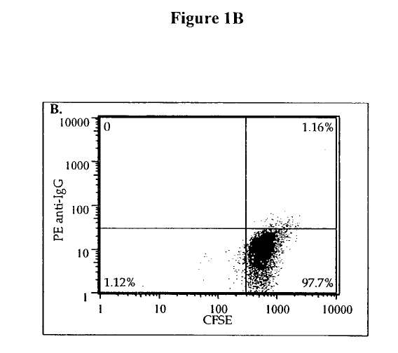

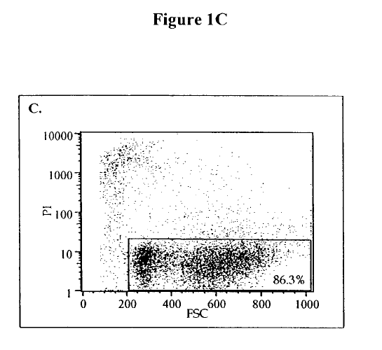

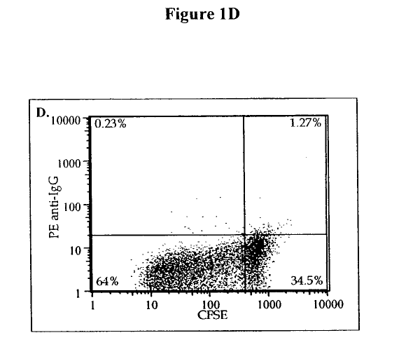

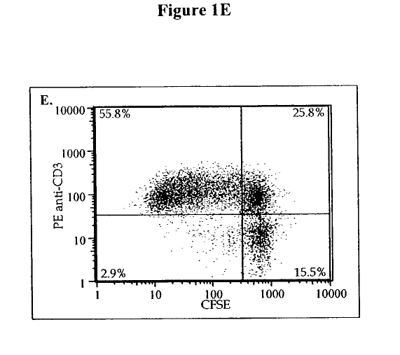

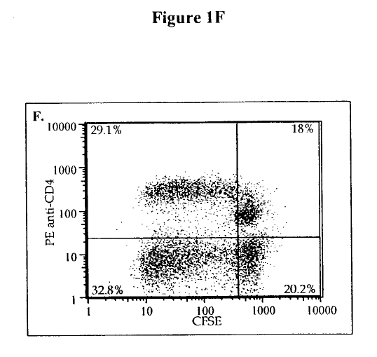

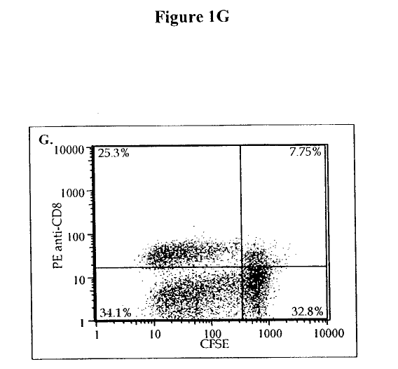

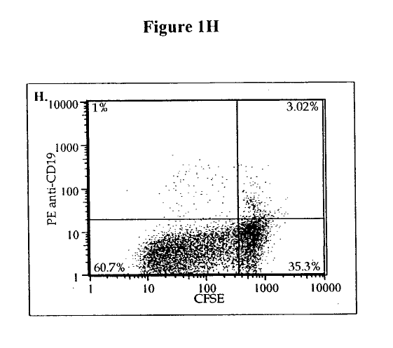

Pan T cell Activation Sample Data: Analysis of Control Cells. Unstimulated human PBL (FIG. 1A and B, respectively) and PHA stimulated (FIG. 1C through H, respectively) human PBL were stained with CFSE using the kits disclosed herein, and cultured for four days. The cells were then harvested and stained with PI and PE-conjugated antibodies. Certain aspects of one embodiment of the methods and compositions of the present invention are explained in further detail by reference to FIGS. 1A-G, as described below.

Briefly, FIG. 1A shows a plot of FSC versus PI. From this plot, the population of live cells can be identified and gated for further analysis. FIG. 1B shows a plot of CFSE versus PE IgG. Examination of the level of CFSE staining in the control cell population gated in FIG. 1A identifies the non-proliferating cell population. The quadrant can be applied to subsequent plots. FIG. 1C shows a plot of FSC staining versus PI. Examination of this plot allows setting a gate on the population of live cells in the stimulated culture (compare to FIG. 1A). This gate was applied to all ensuing plots. FIG. 1D shows a plot of CFSE versus PE anti-IgG. Applying quadrant markers to the data allows background IgG staining to be distinguished from specific antibody staining in subsequent plots. Analysis of the quadrant statistics indicates that 64% of the cells proliferated. FIG. 1E shows a plot of CFSE versus PE anti-CD3. The frequency of proliferating CD3⁺ cells is 55.8%. FIG. 1F shows a plot of CFSE versus PE anti-CD4. The frequency of proliferating CD4⁺ cells is 29.1%. FIG. 1G shows a plot of CFSE versus PE anti-CD8. The frequency of proliferating CD8⁺ cells is 25.3%. FIG. 1H shows a plot of CFSE versus PE anti-CD19. The frequency of proliferating CD19⁺ cells is 1%.

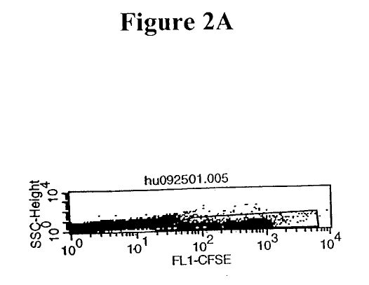

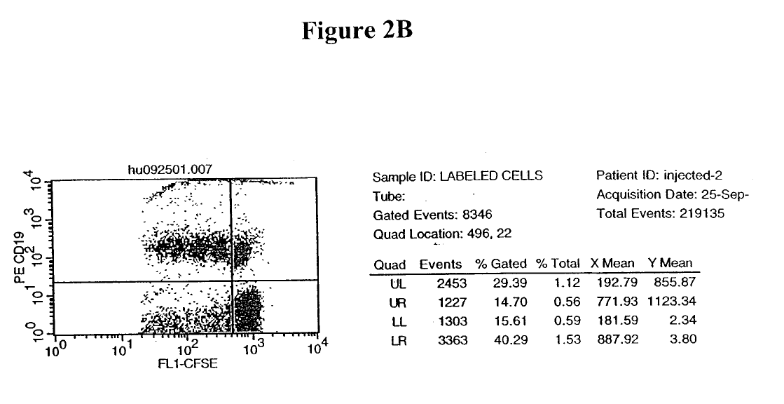

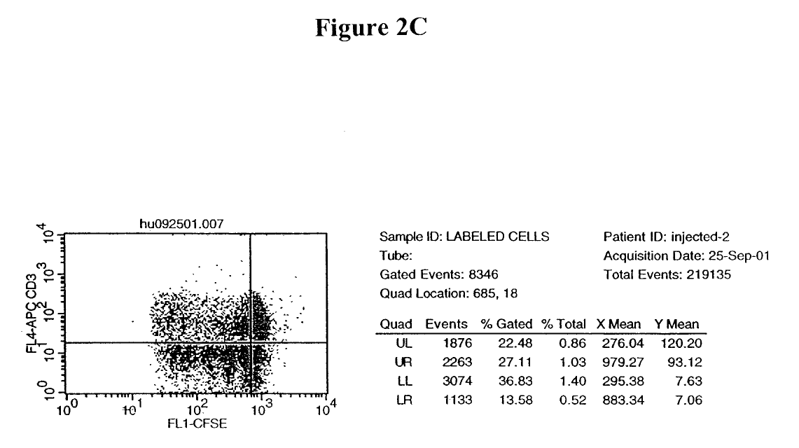

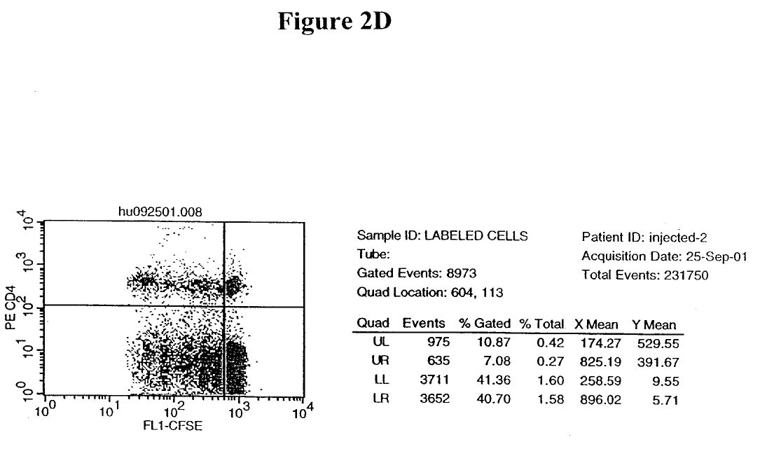

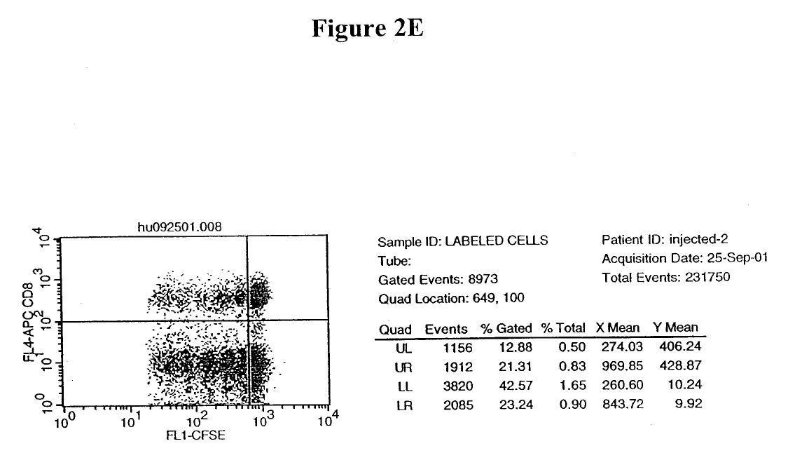

Additional examples of the flow cytometry data acquisition and analysis features of the present invention discussed above are presented in FIGS. 2A-2E. Briefly, each of FIGS. 2A-2E provide example of the user partitioned data sets (e.g., gated sets) on flow cytometry data analysis. FIG. 2A shows the raw data stream from a flow cytometry assay experiment. In FIG. 2A the user partitions the data filed into a first and second region. It is contemplated that this initial partitioning substantially excludes background signal from signal generated by cells of interest. Thus, the initial partitioning step is contemplated to substantially improve the accuracy of subsequent data filed partitions and manipulations. Each of FIGS. 2B-2E show with increasing accuracy the effects of user defined sets and the ability of this technique to home in on data from the cells of interest.

Exemplary Staining Protocols

Preferred embodiments of the present invention using the compositions and method disclosed herein are provided below as a non-limiting description of the present invention.

In a preferred embodiment, a kit is supplied comprising: carboxy-fluorescein diacetate, succinimidyl ester (CFSE) provided in single use pre-aliquoted tube(s). The pre-aliquoted tubes of CFSE are stored at −20° C. As stated above, the present invention contemplates that CFSE passively diffuses into cells and is converted into a fluorescent carboxyfluorescein succinimidyl ester by resident intracellular esterases. During each round of cell division, the CFSE fluorescence is halved, thus allowing for the identification of successive cell generations. CFSE is detected using standard fluorescein filters (excitation=492 nm, emission=517 nm) using standard flow cytometry detection techniques. In certain embodiments directed to human cell proliferation studies, the kit further comprises phytohemagglutinin (PHA). PHA is an extract of red kidney bean that induces the global activation of T cells. In preferred embodiments, the PHA is provided in a pre-aliquoted tube(s). Certain of these embodiments use 10 μl of the pre-aliquoted PHA stock solution per 10 ml of medium. The PHA should be stored at −20° C. In certain other embodiments directed to murine cell proliferation studies, the kit substitutes or further provide concanavalin A (Con A). Con A is a mitogen that activates murine T cells. In certain of these embodiments, Con A is provided in a pre-aliquoted portion(s) at a concentration of 1 mg/mL. The present invention contemplates that the Con A is used at a final concentration of 5 μg/ml. The aliquots of Con A are stored at −20° C.

In certain preferred embodiments, the kits further comprise pre-aliquoted portions of propidium iodide (PI) staining solution. In certain of these embodiments, the pre-aliquoted portion of PI provides enough reagent for about 20 tests. The pre-aliquoted tube of PI is stored at 4° C. PI stains the DNA of cells that have lost cell membrane integrity. Dead cells, or cells in late stages of apoptosis, are permeable to PI. Detection is carried out using 562-588 nm band pass filter.

In still other embodiments, the kits further comprise one or more pre-aliquoted portions of R-Phycoerythrin (R-PE) conjugated antibodies. In certain of these embodiments, the R-PE antibodies are selected from the group comprising: R-PE anti IgG; R-PE anti CD3; R-PE anti CD4; R-PE anti CD8, and R-PE anti CD19. The R-PE conjugated antibodies are stored in the dark at 4° C. The present invention contemplates that about 3 μl of one R-PE antibody solution is added to each staining reactions as required. Various other reagents, culture media, and buffers further comprise additional embodiments of the present invention as described in greater detail above.

It should be appreciated that the kits disclosed herein are not intended to be limited to providing only one pre-aliquoted tube or portion of a particular reagent or compound supplied therein (e.g., 1 aliquot of reagent X, 1 aliquot of reagent Y, etc.). Indeed, the present invention contemplates that kits comprising numerous single aliquots portions of any of the reagents are within the scope of the present invention (e.g., 2 or more aliquots of reagent X, 2 or more aliquots of reagent Y, etc.).

Exemplary Human Cell Proliferation Protocol

The following human T-cell CFSE staining protocol is provided as a non-limiting exemplary description of one human cell proliferation study contemplated by the present invention.

I. Fluorescent Labeling of Cells

A. Prepare cells for staining.

1. Resuspend 5×10⁶ human lymphocytes in 1 ml of PBS pH 7.2-7.4.

B. Label cells.

1. Add resuspended cells to a tube containing CFSE.

2. Incubate cells for 10 min at 37 C mixing twice.

3. Transfer cells to a 15 ml conical tube containing 1 ml ice cold culture medium and mix gently for 1 min.

C. Remove unincorporated CFSE.

1. Add 10 ml PBS to the tube. Centrifuge cells for 10 min at 1500 rpm and decant supernatant.

2. Wash cells once more as above.

II. Pan T Cell Activation for Human Lymphocytes

A. Preparation of experimental culture: resuspend 4×10⁶ cells in 10 ml culture medium. Add 10 μL PHA.

B. Preparation of control culture: resuspend 1×10⁶ CFSE labeled Cells in 5 ml of culture medium.

C. Transfer the cells to separate culture flasks and incubate for 4 days at 37 C in a humidified incubator containing 5% CO₂.

III. Mixed Lymphocyte Reaction

A. Preparation of stimulator cells: inactivate 4×10⁶ stimulator cells by irradiation or treatment with Mitomycin C. Wash the cells 3 times as in step I. C.

B. Preparation of experimental culture: resuspend 4×10⁶ CFSE stained responder cells and mix with 4×10⁶ stimulator cells in a final volume of 10 ml culture medium.

C. Preparation of control culture: resuspend 1×10⁶ CFSE labeled responder cells in 5 ml culture medium.

D. Transfer the cells to separate culture flasks and incubate for 4 days at 37 C in humidified incubator containing 5% CO₂.

IV. Immunofluorescent Staining of Cell Surface Molecules.

A. Prepare cells: transfer the cultured cells to separate 15 ml conical tubes and spin cells at 1500 rpm for 10 min. Resuspend cells in 1 ml of PBS.

1. Add approximately 0.5-1×10⁶ of cells from the experimental culture to each of five 12×75 mm tubes. Add approximately 0.5-1×10⁶ of cells from the control culture to two 12×75 mm tubes.

2. Wash cells once with 3 mL PBS as in step I. C. Resuspend cells in 200 μl PBS.

B. Immunofluorescent staining of cell surface antigens

1. Staining of control cells: add 3 μl control anti-IgG or anti-CD3 antibody to each tube.

2. Staining of experimental cells: add 3 μl of control IgG, anti-CD3, anti-CD4, anti-CD8, or anti-CD19 antibody to each tube.

3. Incubate on ice for 20 min in the dark.

4. Wash once with 2 ml PBS as in step I. C.

V. Resuspension of Cells

A. Add 500 ml PBS to each tube to resuspend cells.

VI. Staining of Cells with Propidium Iodide (PI)

A. Add 5 ml of PI to each tube prior to data acquisition by flow cytometer.

VII. Perform Flow Cytometric Analysis

A. Acquire data using flow cytometry and acquire multiparameter files.

B. Collect green fluorescence (CFSE) with a 525 nm band pass filter.

C. Collect orange fluorescence (PE) with a 575 nm band pass filter.

Exemplary Murine Cell Proliferation Protocol

The following murine T-cell CFSE staining protocol is provided as a non-limiting exemplary description of one murine cell proliferation study contemplated by the present invention.

I. Fluorescent Labeling of Cells

A. Prepare cells for staining.

1. Resuspend murine 5×10⁶ lymphocytes in 1 ml of PBS pH 7.2-7.4.

B. Label cells.

1. Add resuspended cells to a tube containing CFSE.

2. Incubate cells for 10 min at 37 C mixing twice.

3. Transfer cells to a 15 ml conical tube containing 1 ml ice cold culture medium and mix gently for 1 min.

C. Remove unincorporated CFSE.

1. Add 10 ml PBS to the tube. Centrifuge cells for 10 min at 1500 rpm, and decant supernatant.

2. Wash cells once more as above.

II. Pan T Cell Activation for Murine Lymphocytes

A. Preparation of experimental culture: resuspend 4×10⁶ CFSE labeled cells in 10 ml culture medium. Add 50 mL of the provided Con A for a final concentration of 5 μg/ml.

B. Preparation of control culture: resuspend 1×10⁶ CFSE labeled cells in 5 ml of culture medium.

C. Transfer cells to separate culture flasks and incubate for 4 days at 37 C in a humidified incubator containing 5% CO₂.

III. Mixed Lymphocyte Reaction

A. Preparation of stimulator cells: inactivate 4×10⁶ stimulator cells by irradiation or treatment with Mitomycin C. Wash the cells 3 times as in step I. C.

B. Preparation of experimental culture: resuspend 4×10⁶ CFSE stained responder cells and mix with 4×10⁶ stimulator cells in a final volume of 10 ml culture medium.

C. Preparation of control culture: resuspend 1×10⁶ CFSE labeled responder cells in 5 ml culture medium.

D. Transfer the cells to separate culture flasks and incubate for 4 days at 37 C in humidified incubator containing 5% CO₂.

IV. Immunofluorescent Staining of Cell Surface Molecules.

A. Prepare cells: transfer the cultured cells to separate 15 ml conical tubes and spin cells at 1500 rpm for 10 min. Resuspend cells in 1 ml of PBS.

1. Add approximately 0.5-1×10⁶ of cells from the experimental culture to each of five 12×75 mm tubes. Add approximately 0.5-1×10⁶ of cells from the control culture to two 12×75 mm tubes.

2. Wash cells once with 3 mL PBS as in step I. C. Resuspend cells in 200 μl PBS.

B. Immunofluorescent staining of cell surface antigens

1. Staining of control cells: add 3 μl control anti-IgG or anti-CD3 antibody to each tube.

2. Staining of experimental cells: add 3 μl of control anti-IgG, anti-CD3, anti-CD4, anti-CD8, or anti-CD19 antibody to each tube.

3. Incubate on ice for 20 min in the dark.

4. Wash once with 2 ml PBS as in step I. C.

V. Resuspension of Cells

A. Add 500 ml PBS to each tube to resuspend cells.

VI. Staining of Cells with Propidium Iodide (PI)

A. Add 5 ml of PI to each tube prior to data acquisition by flow cytometer.

VII. Perform Flow Cytometric Analysis

A. Acquire data using flow cytometry and acquire multiparameter files.

B. Collect green fluorescence (CFSE) with a 525 nm band pass filter.

C. Collect orange fluorescence (PE) with a 575 nm band pass filter.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A kit comprising: a container having an aliquot of CFSE dye at a concentration of from 0.8 mM to 1 mM.

2. The kit of claim 1, further comprising two or more R-phycoerythrin conjugated antibodies.

3. The kit of claim 2, wherein said two or more R-phycoerythrin conjugated antibodies are selected from the group consisting of R-PE anti IgG, R-PE anti CD3, R-PE anti CD4, R-PE anti CD8, and R-PE anti CD19.

4. The kit of claim 1, further comprising instructions for using said CFSE dye in a cell proliferation assay experiment.

5. The kit of claim 1, wherein said aliquot of CFSE dye contains an amount of said CFSE dye suitable for a single cell proliferation assay experiment.

6. The kit of claim of 1, further comprising a container having an aliquot of propidium iodide staining solution.

7. The kit of claim 6, wherein said aliquot of said propidium iodide staining solution contains an amount of said propidium iodide staining solution suitable for about 20 or more cell proliferation assay experiments.

8. The kit of claim 1, further comprising a container having an aliquot of phytohemagglutinin.

9. The kit of claim 1, further comprising a container having an aliquot of concanavalin A.

10. The kit of claim 1, wherein said container having an aliquot of CFSE dye at a concentration of from 0.08 mM to 1 mM is stable for at least 7 months.

11. The kit of claim 1, further comprising one or more compounds for detecting intracellular compounds of interest.

12. The kit of claim 11, wherein said intracellular compounds of interest are selected from the group consisting of cytokines and chemokines.

13. A kit for conducting cell proliferation assays comprising:
a) a first container having an aliquot of first fluorescein dye, wherein said fluorescein dye comprises CFSE dye at a concentration of from 0.8 mM to 1 mM; and
b) a second container having an aliquot of a second fluorescein dye.

14. The kit of claim 13, further comprising instructions for using said first CFSE dye and said second fluorescein dye in a cell proliferation assay experiment.

15. The kit of claim 13, wherein said aliquot of CFSE dye contains an amount of said CFSE dye suitable for a single cell proliferation assay experiment.

16. The kit of claim 13, wherein said two or more R-phycoerythrin conjugated antibodies are selected from the group consisting of R-PE anti IgG, R-PE anti CD3, R-PE anti CD4, R-PE anti CD8, and R-PE anti CD19.

17. The kit of claim of 13, further comprising a container having an aliquot of propidium iodide staining solution.

18. The kit of claim 17, wherein said aliquot of said propidium iodide staining solution contains an amount of said propidium iodide staining solution suitable for about 20 or more cell proliferation assay experiments.

19. The kit of claim 13, further comprising a container having an aliquot of phytohemagglutinin.

20. The kit of claim 13, further comprising a container having an aliquot of concanavalin A.

21. The kit of claim 13, wherein said a container having an aliquot of CFSE dye at a concentration of from 0.8 mM to 1 mM is stable for at least 7 months.

22. A kit comprising:
a) a first container having an aliquot of first fluorescein dye, wherein said fluorescein dye comprises CFSE dye at a concentration of from 0.8 mM to 1 mM;
b) a second container having an aliquot of a second fluorescein dye;
c) two or more R-phycoerythrin conjugated antibodies; and
d) instructions for using said first CFSE dye and said second fluorescein dye in a cell proliferation assay experiment.

23. The kit of claim 22, wherein said aliquot of CFSE dye contains an amount of said CFSE dye suitable for a single cell proliferation assay experiment.

24. The kit of claim 22, wherein said two or more R-phycoerythrin conjugated antibodies are selected from the group consisting of R-PE anti IgG, R-PE anti CD3, R-PE anti CD4, R-PE anti CD8, and R-PE anti CD19.

25. A composition comprising a container having an aliquot of CFSE dye at a concentration of from 0.8 mM to 1 mM, wherein said aliquot of CFSE dye contains an amount of said CFSE dye suitable for a single cell proliferation assay experiment, wherein said aliquot of said CFSE dye is stable for at least 7 months.

26. A method of data analysis comprising:
a) providing data from a flow cytometry assay experiment, wherein said data is displayed such that data corresponding to test cells are distinguishable from data corresponding to background cells;
b) selecting a subset of said data from flow cytometric assay experiment such that said subset contains substantially all of said data corresponding to test cells and excludes substantially all of said data corresponding to said background cells; and
c) analyzing said subset to provide a cell proliferation result.

27. The method of claim 26, wherein said analyzing comprises preparing one or more user defined gated sets of said data from a flow cytometry assay experiment, wherein said one or more user defined gated sets of said data from a flow cytometry assay experiment comprises a first, second, third, and fourth regions.

28. The method of claim 27, wherein said analyzing comprises determining the ratio of said one or more of said first, second, third, and fourth regions of said user defined gated sets of said data from flow cytometry assay experiment.
